SEQUENCE LISTING (4 sequences)

SEQ ID NO: 1 - protein, 119 aa, Artificial Sequence
  CV1
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 2 - protein, 18 aa, Artificial Sequence
  CD47 signal peptide
MWPLVAALLLGSACCGSA

SEQ ID NO: 3 - protein, 142 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNEN

SEQ ID NO: 4 - protein, 124 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNEN

(19) United States
(12) Patent Application Publication
(10) Pub. No.: US 2022/0235131 A1
(43) Pub. Date: Jul. 28, 2022

(54) METHODS OF TREATING INFECTIONS BY BLOCKING PATHOGEN MIMICS OF CD47

(71) Applicant: The Board of Trustees of the Leland Stanford Junior University, Stanford, CA (US)

(72) Inventors: Michal Caspi Tal, Cupertino, CA (US); Irving L. Weissman, Stanford, CA (US); Laughing Bear Torrez Dulgeroff, Stanford, CA (US); Effie Bastounis, Stanford, CA (US); Efthymia Matthalou, Stanford, CA (US); Joe Hsu, Stanford, CA (US); Maxim Markovic, Stanford, CA (US); Maia Shoham, Stanford, CA (US); Balyn Wood Zaro, Stanford, CA (US); Tal Bachar Raveh, Stanford, CA (US)

(21) Appl. No.: 17/617,797

(22) PCT Filed: Jun. 16, 2020
(86) PCT No.: PCT/US2020/037933

§ 371 Date: Dec. 9, 2021

Related U.S. Application Data
(60) Provisional application No. 62/862,492, filed on Jun. 17, 2019.

(51) Int. Cl. C07K 16/28; G01N 33/569

(52) U.S. Cl. CPC C07K 16/2803; G01N 33/56911; G01N 2333/70596; G01N 2333/20; G01N 2333/38; G01N 33/56961

(57) ABSTRACT

Methods are provided for treating a subject for an infection by a pathogen expressing a CD47-like mimic protein on its surface. In particular, the methods comprise administering an agent that reduces the binding of the CD47 mimic protein on the pathogen to SIRPα on a phagocytic cell, wherein the agent is administered at an effective dose for increasing phagocytosis of the pathogen

C

### CROSS REFERENCE

This application claims benefit of U.S. Provision Patent Application No. 62/862,492, filed Jun. 17, 2019, which applications are incorporated herein by reference in their entirety.

### BACKGROUND OF THE INVENTION

Turnover of cells begins with the induction of an apoptotic program or other cellular changes that mark them for removal, and the subsequent recognition of markers by phagocytes, including macrophages, dendritic cells, and the like. This process requires a specific and selective removal of unwanted cells. Unlike healthy cells, the unwanted/aged/dying cells display markers or ligands called “eat-me” signals, i.e. “altered self”, which can in turn be recognized by receptors on the phagocytes. Healthy cells may display “don't eat-me” signals that actively inhibit phagocytosis; these signals are either downregulated in the dying cells, are present in an altered conformation or they are superseded by the upregulation of “eat-me” or pro-phagocytic signals. The cell surface protein CD47 on healthy cells and its engagement of a phagocyte receptor, signal regulatory protein a (SIRPα), constitutes a key “don't eat-me” signal that can turn off engulfment mediated by multiple modalities, including apoptotic cell clearance and FcR mediated phagocytosis. Blocking the CD47 mediated engagement of SIRPα on a phagocyte, or the loss of CD47 expression in knockout mice, can cause removal of live cells and non-aged erythrocytes. Alternatively, blocking SIRPα also allows engulfment of targets that are not normally phagocytosed, for those cells where pre-phagocytic signals are also present.

CD47 is a broadly expressed transmembrane glycoprotein with a single Ig-like domain and five membrane spanning regions, which functions as a cellular ligand for SIRPα with binding mediated through the NH2-terminal V-like domain of SIRPα. SIRPα is expressed primarily on myeloid cells, including macrophages, granulocytes, myeloid dendritic cells (DCs), mast cells, and their precursors, including hematopoietic stem cells. Structural determinants on SIRPα that mediate CD47 binding are discussed by Lee et al. (2007) J. Immunol. 179:7741-7750; Hatherley et al. (2007) J.B.C. 282:14567-75; and the role of SIRPα cis dimerization in CD47 binding is discussed by Lee et al. (2010) J.B.C. 285:37953-63.

In keeping with the role of CD47 to inhibit phagocytosis of normal cells, there is evidence that it is transiently upregulated on hematopoietic stem cells (HSCs) and progenitors just prior to and during their migratory phase, and that the level of CD47 on these cells determines the probability that they are engulfed in vivo. CD47 is also constitutively upregulated on a number of cancers. Overexpression of CD47 by tumor cells may increase pathogenicity by allowing cancerous cells to evade phagocytosis.

Programmed cell death (PCD) and phagocytic cell removal are common ways that damaged, precancerous, inflamed, or infected cells respond to pathogenic threats to the organism. However, some infections persist for long periods of time, suggesting that successful persistent infections overcome the PCD and phagocytic cell removal pathways.

There remains a need for better methods of treating infections to overcome pathogen avoidance of innate immune responses.

### SUMMARY OF THE INVENTION

Methods are provided for treating an individual infected with a pathogen comprising a pathogenic CD47 mimic protein by administering an effective dose of an agent that blocks the CD47 mimic protein present on the pathogen, wherein the dose of the agent is effective in decreasing infection by the pathogen. In some embodiments, the pathogen is a Borrelia or Aspergillus pathogen. A Borrelia pathogen comprising a pathogenic CD47 mimic protein may include, without limitation, Borrelia burgdorferi. An Aspergillus pathogen comprising a pathogenic CD47 mimic protein may include, without limitation, i Aspergillus fumigatus, Aspergillus versicolor, Aspergillus flavus, Aspergillus niger, Aspergillus terreus, and Aspergillus nidulans. In some embodiments, the agent binds to the CD47 mimic protein on Aspergillus conidia.

In some embodiments, a method of inhibiting an infection of a subject by a pathogen comprising a pathogenic CD47 mimic protein comprising administering to the subject an effective amount of an agent that reduces binding of the CD47 mimic protein on the pathogen to a signal regulatory protein a (SIRPα) on a phagocytic cell.

Suitable agents for decreasing infection include, without limitation, SIRPα polypeptides, which include soluble high affinity SIRPα polypeptides; antibodies specific for the CD47 mimic protein on the pathogen, and the like. A high affinity SIRPα polypeptide may comprise amino acid substitutions at one or more, two or more, three or more, four or more, five or more, and not more than 14 amino acids within the combined set of contact residues and the set of hydrophobic core residues in the d1 domain of SIRPα, wherein the amino acid modification increases the affinity of the SIRPα polypeptide binding to CD47. For example, amino acid modifications may be made at wherein amino acid modifications are made at one or more of the amino acids within the set that includes, without limitation, residues L4, V6, A21, V27, I31, E47, K53, E54, H56, S66, K68, V92, F94, and F103, or a combination thereof, and may be one or more of (1) L4V; L4I; (2) V6I; V6L; (3) A21V; (4) V27I; V27L; (5) I31T; I31S; I31F; (6) E47V; E47L; (7) K53R; (8) E54Q; (9) H56P; H56R; (10) S66T; S66G; (11) K68R; (12) V92I; (13) F94L; F94V; (14) V63I; and (15) F103V, as described in U.S. Pat. No. 9,944,911. In some embodiments, the agent that binds to the CD47 mimic protein comprises a SIRPα polypeptide such as, but not limited to, CV1 or FD6. In some embodiments, the SIRPα polypeptide is fused to an Fc domain of an antibody including, without limitation, IgG1, IgG2, IgG3, IgG4, IgA, IgE, IgD, and IgM.

In some embodiments, the subject that is treated by the methods described herein is a mammalian subject, including without limitation, a human, dog, cat, pig, sheep, cow, goat, horse, non-human primate, etc.

In some embodiments the methods are used for treating a bacterial or fungal infection, wherein the pathogen comprises a CD47 mimic protein. In some embodiments, the agent is conjugated to an antibacterial agent or an antifungal agent.

In some embodiments, the methods provided are used for targeting or depleting the pathogen, comprising contacting an infected biological sample, e.g. blood from an infected subject, with an agent that specifically binds to the CD47 mimic protein, in order to target or deplete the pathogen. In certain aspects, the agent is an antibody specific for the CD47 mimic protein or a high affinity soluble SIRPα conjugated to an antimicrobial agent, antifungal agent, or cytotoxic agent, e.g., radioactive isotope, chemotherapeutic agent, toxin, etc.

In another aspect, a method of detecting a pathogen expressing a pathogenic CD47 mimic protein is provided, the method comprising: contacting the CD47 mimic protein on the pathogen with a probe comprising detectably labeled CV1-G4, wherein the detectably labeled CV1-G4 binds to the CD47 mimic protein to form a complex; and detecting a signal from the detectably labeled CV1-G4 in the complex. In some embodiments, the pathogen is a Borrelia or an Aspergillus pathogen.

In some embodiments, the probe comprises a detectable label including, without limitation, a fluorophore, a chemiluminescent label, a bioluminescent label, an isotopic label, or a contrast agent.

In some embodiments, the probe is immobilized on a solid support including, for example, without limitation, a magnetic bead, a non-magnetic bead, a membrane, or a gel.

In some embodiments, the method is performed in vivo or in vitro.

In some embodiments, the method further comprises isolating the pathogen from the complex.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1B show that Bb express a CD47 mimic, and treatment with CV1-G4 increases macrophage phagocytosis. FIG. 1A: Bb express a mimic of human CD47 and bind CV1-G4. Bb-GFP-GFP-expressing Bb, Bb OSP-Bb stained with a polyclonal antibody to Bb outer surface proteins (OSP), CV1-Bb stained with CV1-G4 directly conjugated to APC. FIG. 1B: Human monocyte-derived macrophages were incubated with Bb-GFP and treated with either an IgG4 isotype control antibody or CV1-G4 at 10 μg/ml; phagocytosis was improved with CD47 blockade (CV1).

FIGS. 2A-2B show that isotype profiling demonstrates that CV1-G4 coating of Bb-GFP leads to augmented IgG2a responses and decreases IgE responses. C3H mice were infected with Bb-GFP and monitored for 67 days. Serum from different infection conditions was incubated with Bb in culture and the levels of Bb-specific antibodies were quantified. FIG. 2A: Graphs of Bb-specific percentages of IgG2a and IgE antibodies. IgG2a is the murine equivalent of human IgG1, and is the most activating IgG subtype. FIG. 2B: Representative FACS plots are shown with IgG2a and IgE levels at day 67 post-infection for a) Bb alone; b) serum from mice infected with Bb in BSK media; c) serum from mice infected with Bb in CHO media; d) serum from mice infected with Bb in CHO media and CV1-G4; e) uninfected serum. These data show that CV1-G4 causes a shift in the immune response towards production of antibody subtypes that are favorable for immune clearance of the pathogen.

FIGS. 3A-3C show proteomic approaches for the identification of therapeutic targets of Lyme disease. FIG. 3A: A higher percentage of Bb bind with CV1-G4 when cultured at 37° C. compared to 33° C. B31-GFP Bb were thawed from low passage stocks into 50 mL of BSK-II media complete with 6% rabbit serum and cultured at either 33° C. or 37° C. for 1 week to exponential growth phase. FIG. 3B: Differential protein expression of <400 Bb cultured at 37° C. vs. 33° C. Bb were lysed, protein concentration normalized and lysate subjected to trypsin digestion, cysteine reduction and alkylation. Peptides were then labelled with a isotopically ‘heavy’ or ‘light’ mass tag. Tagged peptides were combined, desalted and subjected to mass spectrometry analysis. The larger the enrichment ratio, the more heavily expressed at 37° C. compared to 33° C. Enrichment ratios below indicate proteins more heavily expressed at 33° C. FIG. 3C: Proteomic workflow for comparing protein expression between discrete spirochetes, round bodies, and Bb immune complex clumps. All three structures of Bb will be purified and lysed. Lysate will be normalized and digested with trypsin. Peptides will then be tagged with light (spirochete), medium (round body) or heavy (complex clumps) mass tags. Once peptides from each stage of infection are tagged, the samples will be combined and subjected to quantitative proteomics analysis.

FIGS. 4A-4B show that CV1-G4 increases macrophage phagocytosis of Borrelia burgdorferi. FIG. 4A shows Human monocyte-derived macrophages after incubation with Bb-GFP which were double-labeled with acid-sensitive pHrodo Red. Bb-GFP can be seen in green. In red are Bb-GFP which have been exposed to the acidic lysosome within the macrophage, and in grey are macrophages. The large image is the original merged image. The top set of images are treated with an IgG4 isotype control antibody at 10 μg/ml, and the set below are treated with CV1-G4 at 10 μg/ml. FIG. 4B shows a quantification of 20 images showing the efficiency of phagocytosis of Bb that were neutralized as a percentage of all Bb.

FIG. 5A-5E. C57B/6 mice were infected IP with 10⁵ Bb-GFP coated in CV1-G4 or IgG4 isotype control and compared to mice injected with Bb-GFP alone at 10⁵ or 10⁴. Serum from these mice was examined 1, 3, and 5 weeks post-infection and antibody subtypes specific for Bb were measured. Mice infected with Borrelia coated in CV1G4 were less likely to get infected and more likely to clear the Bb if they did get infected.

FIGS. 6A-6C show that the CV1-G4 coating of Bb-GFP leads to augmented IgG2a responses and decreases the IgE responses induced by incubating Bb in CHO media. C3H mice were infected with Bb-GFP and monitored over the course of 67-days. Ankles were measured with calipers; peak ankle swelling was observed on day 48 and antibody levels were measured at the day 67 endpoint. FIG. 6A shows ankle width in millimeters by infection condition, showing that the highest degree of swelling and joint-level inflammation was in the group infected with Bb injected in CHO media. Serum from this condition was then incubated with Bb in culture at a 10% concentration as described in FIGS. 1 and 2 to quantify the levels of Bb-specific antibodies. IgG2a (FIG. 6B) and IgE (FIG. 6C) antibody levels at day 67 post-infection for each mouse in the cohort, as well as the ankle widths at peak swelling.

FIGS. 7A-7D show that even with a very high Aspergillus fumigatus infection dose there is a trend towards reduced fungal burden with use of the CV1 coating. FIG. 7A shows Aspergillus fumigatus invasion grading (TL: tracheal lumen, C: cartilage, EL: endothelial layer). FIG. 7B shows the effect of the CV1 coating at 3 days post infection with Aspergillus fumigatus conidia (spores). FIG. 7C shows a control at 3 days post infection with Aspergillus fumigatus conidia (spores) without the CV1 coating. FIG. 7D shows a plot comparing the average fungal burden (a.u.) in the presence and absence of the CV1 coating.

FIGS. 8A-8E shows that Aspergillus fumigatus conidia (spores) have high expression of a CD47 mimic that is binding to CV1-G4 (well above isotype control and secondary antibody only control). As Aspergillus grows into hyphae it loses expression of the CD47 mimic that is binding to CV1-G4. FIGS. 8A-8C show representative FACS plots of binding of CV1-G4 and IgG4 to Aspergillus fumigatus conidia compared to a control. Plots summarizing binding data for Aspergillus fumigatus conidia (FIG. 8D) and hyphae (FIG. 8E) are also shown.

FIGS. 9A-9B show that Aspergillus fumigatus conidia, not hyphae, bind CV1. FIG. 9A shows a plot of the percentage of CV1 positive Aspergillus fumigatus conidia (spores) and hyphae. FIG. 9B shows a schematic of the life cycle of Aspergillus fumigatus.

FIGS. 10A-10B shows FACS analysis of binding of CV1-G4 to Aspergillus fumigatus conidia (FIG. 10A) and hyphae (FIG. 10B).

FIGS. 11A-11B show CV1 binding among Aspergillus strains. FIG. 11A shows screening of Aspergillus strains for CV1 binding FIG. 11B shows table summarizing CV1 binding data for various Aspergillus strains.

FIGS. 12A-12B show lack of CV1 binding in other fungi and Pseudomonas aeruginosa. FIG. 12A shows CV1 binding to selected Candida, Rhizopus, and Pseudomonas pathogens of interest. FIG. 12B shows representative FACS analysis of CV1 staining.

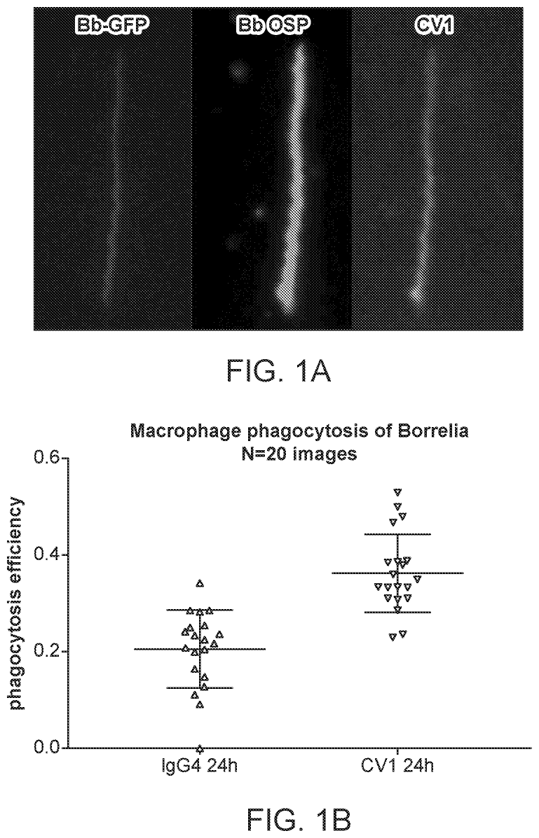

### DETAILED DESCRIPTION OF THE INVENTION

The present invention relates to methods of treating a subject for an infection by administering an agent that reduces the binding of a CD47 mimic protein on a pathogen to SIRPα on a phagocytic cell, which may be referred to herein as an anti-CD47 mimic agent.

The terms “treatment”, “treating”, “treat” and the like are used herein to generally refer to obtaining a desired pharmacologic and/or physiologic effect. The effect can be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom. Those in need of treatment include those already with an infection as well as those in which an infection is to be prevented. As such, a therapeutic treatment is one in which the subject is infected prior to administration and a prophylactic treatment is one in which the subject is not infected prior to administration. In some embodiments, the subject is suspected of being infected prior to administration. In some embodiments, the subject has an increased risk of infection prior to administration. In some embodiments, the subject is suspected of being at increased risk of infection prior to administration.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

An “effective amount” is an amount sufficient to effect beneficial or desired clinical results in treatment of an infection. By “effective amount” is intended an amount of an anti-CD47 mimic agent that is sufficient to palliate, ameliorate, stabilize, reverse, prevent, slow or delay the progression of a disease state (e.g., infection) by increasing phagocytosis of a pathogen expressing a pathogenic CD47 mimic protein. An effective amount can be administered in one or more administrations.

As used herein, a “target pathogen” is a pathogen expressing a CD47 mimic protein on its surface. Administration of an anti-CD47 mimic agent (e.g., agent that reduces the binding of a CD47 mimic protein on a pathogen to SIRPα on a phagocytic cell) results in increased phagocytosis of the target pathogen. The target pathogen may include, but is not limited to bacteria, viruses, protozoans, and fungi that express a CD47 mimic protein. The pathogen may be an intracellular or extracellular pathogen. Infectious diseases that can be treated by the methods described herein are disorders caused by infectious pathogens comprising a CD47 mimic protein. Some infectious agents may cause no recognizable symptoms or disease under certain conditions, but have the potential to cause symptoms or disease under changed conditions.

As used herein, the term “anti-CD47 mimic agent” refers to any agent that reduces the binding of a CD47 mimic protein (e.g., on an infectious pathogen) to SIRPα (e.g., on a phagocytic cell). Non-limiting examples of suitable anti-CD47 mimic reagents include high affinity SIRPα reagents, anti-SIRPα antibodies, soluble CD47 polypeptides, and antibodies or antibody fragments specific for the CD47 mimic protein. In some embodiments, a suitable anti-CD47 mimic agent (e.g. an antibody specific for the anti-CD47 mimic, a high affinity SIRPα reagent, etc.) binds to a CD47 mimic protein to reduce the binding of the CD47 mimic protein to SIRPα. In some embodiments, a suitable anti-CD47 mimic agent (e.g., an anti-SIRPα antibody, a soluble CD47 polypeptide, etc.) specifically binds SIRPα to reduce the binding of the CD47 mimic protein to SIRPα. A suitable anti-CD47 mimic agent that binds SIRPα does not activate SIRPα (e.g., in the SIRPα-expressing phagocytic cell). The efficacy of a suitable anti-CD47 mimic agent can be assessed by assaying the agent (further described below). In an exemplary assay, a pathogen comprising a pathogenic CD47 mimic protein is incubated in the presence or absence of the candidate agent. An agent for use in the methods of the invention will up-regulate phagocytosis by at least 10% (e.g., at least 20%, at least 30%, at least 40%, at least 50%, at least 60%, at least 70%, at least 80%, at least 90%, at least 100%, at least 120%, at least 140%, at least 160%, at least 160%, or at least 200%) compared to phagocytosis in the absence of the agent. Similarly, an in vitro assay for levels of tyrosine phosphorylation of SIRPα will show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the candidate agent.

In some embodiments, the anti-CD47 mimic agent does not activate CD47 upon binding. When CD47 is activated, a process akin to apoptosis (i.e., programmed cell death) occurs (Manna and Frazier, Cancer Research, 64, 1026-1036, Feb. 1, 2004). Thus, in some embodiments, the anti-CD47 mimic agent does not directly induce cell death of a CD47-expressing cell.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides, or high affinity binding of a SIRPα polypeptide). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member). Suitable specific binding members include agents that specifically bind to a CD47 mimic protein (i.e., anti-CD47 mimic agents), or that otherwise block the interaction between a CD47 mimic protein and SIRPα.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972)Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 1496:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule. Included, for example, is the use of magrolimab.

In one embodiment, the anti-CD47 mimic agent, or a pharmaceutical composition comprising the agent, is provided in an amount effective to detectably inhibit the binding of a CD47 mimic protein on a pathogen to SIRPα present on the surface of phagocytic cells. The effective amount is determined via empirical testing routine in the art, for example in a biological sample taken from an infected individual. The effective amount may vary depending on the number of cells being targeted, the location of the cells, and factors specific to the subject.

The terms “phagocytic cells” and “phagocytes” are used interchangeably herein to refer to a cell that is capable of phagocytosis. There are three main categories of phagocytes: macrophages, mononuclear cells (histiocytes and monocytes); polymorphonuclear leukocytes (neutrophils) and dendritic cells.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cellular pathogens or infected cells. The definition also includes sample that have been enriched for particular types of molecules, e.g., nucleic acids, polypeptides, etc.

The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes an infected sample obtained from a patient infected with a pathogen comprising a pathogenic CD47 mimic protein, e.g., a sample comprising the pathogen or cells infected with the pathogen or polynucleotides and/or polypeptides that are obtained from a patient's infected cell (e.g., a cell lysate or other cell extract comprising polynucleotides and/or polypeptides); and a sample comprising pathogens comprising a CD47 mimic protein. A biological sample comprising a pathogen or an infected cell from a patient can also include non-infected cells.

High affinity SIRPα reagent. In some embodiments, a subject anti-CD47 mimic agent is a “high affinity SIRPα reagent”, which includes SIRPα-derived polypeptides and analogs thereof. High affinity SIRPα reagents are described in U.S. Pat. No. 9,944,911, which is hereby specifically incorporated by reference. High affinity SIRPα reagents are variants of the native SIRPα protein. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide for a CD47 mimic protein, for example by decreasing the off-rate by at least 10-fold, at least 20-fold, at least 50-fold, at least 100-fold, at least 500-fold, or more.

A high affinity SIRPα reagent comprises the portion of SIRPα that is sufficient to bind a CD47 mimic protein at a recognizable affinity, e.g., high affinity, which normally lies between the signal sequence and the transmembrane domain, or a fragment thereof that retains the binding activity. The high affinity SIRPα reagent will usually comprise at least the d1 domain of SIRPα with modified amino acid residues to increase affinity. In some embodiments, the high affinity SIRPα reagent is CV1, which comprises the following amino acid changes relative to the wild-type allele: V6I, V27I, I31F, E47V, K53R, E54Q, H56P, S66T, and V92I. CV1 comprises a variant d1 domain amino acid sequence as follows EEELQIIQPD KSVLVAAGET ATLRCTITSL FPVGPIQWFR GAGPGRVLIY NQRQGPFPRV TTVSDTTKRN NMDFSIRIGN ITPADAGTYY CIKFRKGSPD DVEFKSGAGT ELSVRAKPS (SEQ ID NO:1). In some embodiments, a SIRPα variant is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. In other embodiments, the second polypeptide is any suitable polypeptide that is substantially similar to Fc, e.g., providing increased size, multimerization domains, and/or additional binding or interaction with Ig molecules. In some embodiments, the high affinity SIRPα reagent is a fusion protein comprising CV1 fused to an immunoglobulin Fc region. For example, CV1 may be fused to the Fc region of an immunoglobulin including, without limitation, IgG1, IgG2, IgG3, IgG4, IgA, IgE, IgD, and IgM.

A suitable high affinity SIRPα reagent reduces (e.g., blocks, prevents, etc.) the interaction between SIRPα and a CD47 mimic protein. In some embodiments, the SIRPα reagent amino acid comprise changes that provide for increased affinity, which are localized in the d1 domain. Thus high affinity SIRPα reagents may comprise a d1 domain of human SIRPα comprising at least one amino acid change relative to the wild-type sequence within the d1 domain. Such a high affinity SIRPα reagent optionally comprises additional amino acid sequences, for example antibody Fc sequences; portions of the wild-type human SIRPα protein other than the d1 domain, including without limitation residues 150 to 374 of the native protein or fragments thereof, usually fragments contiguous with the d1 domain; and the like. High affinity SIRPα reagents may be monomeric or multimeric, i.e. dimer, trimer, tetramer, etc.

Anti-CD47 mimic antibodies. In some embodiments, a subject anti-CD47 mimic agent is an antibody that specifically binds the CD47 mimic protein (i.e., an anti-CD47 mimic antibody) and reduces the interaction between the CD47 mimic protein on a pathogen and SIRPα on another cell (e.g., a phagocytic cell). In some embodiments, a suitable anti-CD47 mimic antibody does not activate CD47 upon binding, e.g. Magrolimab.

Anti-SIRPα antibodies. In some embodiments, a subject anti-CD47 mimic agent is an antibody that specifically binds SIRPα (i.e., an anti-SIRPα antibody) and reduces the interaction between the CD47 mimic protein on a pathogen and SIRPα on another cell (e.g., a phagocytic cell). Suitable anti-SIRPα antibodies can bind SIRPα without activating or stimulating signaling through SIRPα because activation of SIRPα would inhibit phagocytosis. Instead, suitable anti-SIRPα antibodies facilitate the phagocytosis of pathogens comprising the pathogenic CD47 mimic protein. Thus, a suitable anti-SIRPα antibody specifically binds SIRPα without activating/stimulating enough of a signaling response to inhibit phagocytosis.

Soluble CD47 polypeptides. In some embodiments, a subject anti-CD47 mimic agent is a soluble CD47 polypeptide that specifically binds SIRPα and reduces the interaction between the CD47 mimic protein on a pathogen and SIRPα on another cell (e.g., a phagocytic cell). A suitable soluble CD47 polypeptide can bind SIRPα without activating or stimulating signaling through SIRPα because activation of SIRPα would inhibit phagocytosis. Instead, suitable soluble CD47 polypeptides facilitate phagocytosis of pathogens comprising the CD47 mimic protein. Those pathogens that express higher levels of the CD47 mimic protein relative to other pathogens (e.g., pathogens that do not express a CD47 mimic protein or at lower levels) will be preferentially phagocytosed. Thus, a suitable soluble CD47 polypeptide specifically binds SIRPα without activating/stimulating enough of a signaling response to inhibit phagocytosis.

In some cases, a suitable soluble CD47 polypeptide can be a fusion protein (for example as structurally described in US Patent Publication US20100239579, herein specifically incorporated by reference). However, only fusion proteins that do not activate/stimulate SIRPα are suitable for the methods provided herein. Suitable soluble CD47 polypeptides also include any peptide or peptide fragment comprising variant or naturally existing CD47 sequences (e.g., extracellular domain sequences or extracellular domain variants) that can specifically bind SIRPα and inhibit the interaction between the CD47 mimic protein and SIRPα without stimulating enough SIRPα activity to inhibit phagocytosis.

In certain embodiments, a soluble CD47 polypeptide comprises the extracellular domain of CD47, including the signal peptide (SEQ ID NO:2), such that the extracellular portion of CD47 is typically 142 amino acids in length, and has the amino acid sequence set forth in SEQ ID NO:3. The soluble CD47 polypeptides described herein also include CD47 extracellular domain variants that comprise an amino acid sequence at least 65%-75%, 75%-80%, 80-85%, 85%-90%, or 95%-99% (or any percent identity not specifically enumerated between 65% to 100%), which variants retain the capability to bind to SIRPα without stimulating SIRPα signaling.

In certain embodiments, the signal peptide amino acid sequence may be substituted with a signal peptide amino acid sequence that is derived from another polypeptide (e.g., for example, an immunoglobulin or CTLA4). For example, unlike full-length CD47, which is a cell surface polypeptide that traverses the outer cell membrane, the soluble CD47 polypeptides are secreted; accordingly, a polynucleotide encoding a soluble CD47 polypeptide may include a nucleotide sequence encoding a signal peptide that is associated with a polypeptide that is normally secreted from a cell.

In other embodiments, the soluble CD47 polypeptide comprises an extracellular domain of CD47 that lacks the signal peptide. In an exemplary embodiment, the CD47 extracellular domain lacking the signal peptide has the amino acid sequence set forth in SEQ ID NO:4 (124 amino acids). As described herein, signal peptides are not exposed on the cell surface of a secreted or transmembrane protein because either the signal peptide is cleaved during translocation of the protein or the signal peptide remains anchored in the outer cell membrane (such a peptide is also called a signal anchor). The signal peptide sequence of CD47 is believed to be cleaved from the precursor CD47 polypeptide in vivo.

In other embodiments, a soluble CD47 polypeptide comprises a CD47 extracellular domain variant. Such a soluble CD47 polypeptide retains the capability to bind to SIRPα without stimulating SIRPα signaling. The CD47 extracellular domain variant may have an amino acid sequence that is at least 65%-75%, 75%-80%, 80-85%, 85%-90%, or 95%-99% identical (which includes any percent identity between any one of the described ranges) to SEQ ID NO:4.

In some cases, an anti-CD47 mimic agent is not a soluble CD47 polypeptide (i.e., is an anti-CD47 mimic agent other than a soluble CD47 polypeptide). In some cases, an anti-CD47 mimic agent binds to SIRPα but is not a soluble CD47 polypeptide (i.e., is a SIRPα binding anti-CD47 mimic agent other than a soluble CD47 polypeptide).

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety and (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used in this invention, the term “epitope” means any antigenic determinant on an antigen to which the paratope of an antibody binds. Epitopic determinants usually consist of chemically active surface groupings of molecules such as amino acids or sugar side chains and usually have specific three-dimensional structural characteristics, as well as specific charge characteristics.

Suitable anti-CD47 antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively.

### METHODS

Methods are provided for treating or reducing infection, including without limitation bacterial, viral, protozoan, and fungal infections, by inhibiting the interaction between SIRPα and a CD47 mimic protein on a pathogen, thereby increasing in vivo phagocytosis of the pathogen. Such methods include administering to a subject in need of treatment a therapeutically effective amount or an effective dose of an anti-CD47 mimic agent, including without limitation combinations of the anti-CD47 mimic agent with another drug.

In some embodiments the infection is a chronic infection, i.e. an infection that is not cleared by the host immune system within a period of up to 1 week, 2 weeks, etc. In some embodiments, the chronic infection is caused by the ability of the pathogen comprising the CD47 mimic protein to evade the immune system by inhibiting phagocytosis. In some cases, chronic infections involve integration of pathogen genetic elements into the host genome, e.g. retroviruses, lentiviruses, Hepatitis B virus, etc. In other cases, chronic infections with, for example certain intracellular bacteria or protozoan pathogens, result from a pathogen cell residing within a host cell. Additionally, in some embodiments, the infection is in a latent stage, as with herpes viruses or human papilloma viruses.

Bacterial pathogens of interest include without limitation, Borrelia pathogens that cause human disease such as Borrelia burgdorferi, Borrelia hermsii, Borrelia miyamotoi, Borrelia afzeffi, Borrelia garinii, Borrelia turicatae as well as other known tick-borne pathogens. Fungal pathogens of interest include without limitation, Aspergillus pathogens such as Aspergillus fumigatus, Aspergillus versicolor, Aspergillus flavus, Aspergillus niger, Aspergillus terreus, and Aspergillus nidulans.

The methods of the invention provide for a more effective removal of pathogens comprising a CD47 mimic protein on their surface by phagocytic cells of the host organism, relative to phagocytosis in the absence of treatment. In some embodiments, the methods of the invention involve diagnosis of a patient as suffering from an infection by a pathogen comprising a CD47 mimic protein; or selection of a patient previously diagnosed as suffering from an infection by a pathogen comprising a CD47 mimic protein; treating the patient with a regimen of anti-CD47 mimic therapy, optionally in combination with an additional therapy; and monitoring the patient for efficacy of treatment. Monitoring may measure clinical indicia of infection, e.g. fever, white blood cell count, etc., and/or direct monitoring for presence of the pathogen.

Treatment may be combined with other active agents. Classes of antibiotics include penicillins, e.g. penicillin G, penicillin V, methicillin, oxacillin, carbenicillin, nafcillin, ampicillin, etc.; penicillins in combination with β-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Cytokines may also be included, e.g. interferon γ, tumor necrosis factor α, interleukin 12, etc. Antiviral agents, e.g. acyclovir, gancyclovir, etc., may also be used in treatment. Antifungal agents such as amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, abafungin, amorolfin, butenafine, naftifine, and terbinafine, anidulafungin, caspofungin, micafungin, and flucytosine may be used in treatment of fungal infections. Steroids may also be used in treatment.

Effective doses of the therapeutic entity of the present invention vary depending upon many different factors, including the nature of the anti-CD47 mimic agent, means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the therapeutic dosage can range from about 0.0001 to 500 mg/kg, and more usually 0.01 to 100 mg/kg, of the host body weight. For example dosages can be 1 mg/kg body weight or 10 mg/kg body weight or within the range of 1-50 mg/kg. The dosage may be adjusted for the molecular weight of the reagent. An exemplary treatment regime entails administration daily, semi-weekly, weekly, once every two weeks, once a month, etc. In another example, treatment can be given as a continuous infusion. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient. It will be understood by one of skill in the art that such guidelines will be adjusted for the molecular weight of the active agent, e.g. in the use of antibody fragments, in the use of antibody conjugates, in the use of high affinity SIRPα reagents, etc. The dosage may also be varied for localized administration, e.g. intranasal, inhalation, etc., or for systemic administration, e.g. i.m., i.p., i.v., and the like.

For the treatment of disease, the appropriate dosage of the anti-CD47 mimic agent will depend on the type of disease to be treated, as defined above, the severity and course of the disease, whether the agent is administered for preventive purposes, previous therapy, the patient's clinical history and response to the antibody, and the discretion of the attending physician. The anti-CD47 mimic agent is suitably administered to the patient at one time or over a series of treatments.

Suitable anti-CD47 mimic agents can be provided in pharmaceutical compositions suitable for therapeutic use, e.g. for human treatment. In some embodiments, pharmaceutical compositions of the present invention include one or more therapeutic entities of the present invention or pharmaceutically acceptable salts, esters or solvates thereof. In some other embodiments, the use of an anti-CD47 mimic agent includes use in combination with another therapeutic agent, e.g., another anti-infection agent. Therapeutic formulations comprising one or more anti-CD47 mimic agents of the invention are prepared for storage by mixing the anti-CD47 mimic agent having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. The anti-CD47 mimic agent composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the anti-CD47 mimic agent to be administered will be governed by such considerations, and is the minimum amount necessary to prevent the CD47 associated disease.

The anti-CD47 mimic agent can be administered by any suitable means, including topical, oral, parenteral, subcutaneous, intraperitoneal, intrapulmonary, and intranasal. Parenteral infusions include intramuscular, intravenous, intraarterial, intraperitoneal, intrathecal or subcutaneous administration. In addition, the anti-CD47 mimic agent is suitably administered by pulse infusion, particularly with declining doses of the agent.

The anti-CD47 mimic agent need not be, but is optionally formulated with one or more agents that potentiate activity, or that otherwise increase the therapeutic effect. These are generally used in the same dosages and with administration routes as used hereinbefore or about from 1 to 99% of the heretofore employed dosages.

An anti-CD47 mimic agent is often administered as a pharmaceutical composition comprising an active therapeutic agent and another pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear an anti-CD47 mimic agent by non-covalent associations, such as non-covalent bonding or by encapsulation. The nature of the carrier can be either soluble or insoluble for purposes of the invention. Those skilled in the art will know of other suitable carriers for binding anti-CD47 mimic agents, or will be able to ascertain such, using routine experimentation.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

Carriers and linkers specific for radionuclide agents include radiohalogenated small molecules and chelating compounds. A radionuclide chelate may be formed from chelating compounds that include those containing nitrogen and sulfur atoms as the donor atoms for binding the metal, or metal oxide, radionuclide.

Radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the methods of the invention. Such moieties may be conjugated to the anti-CD47 mimic agent through an acceptable chemical linker or chelation carrier. Positron emitting moieties for use in the present invention include ¹⁸F, which can be easily conjugated by a fluorination reaction with the anti-CD47 mimic agent.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the anti-CD47 mimic agents can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Mimics of the Mammalian CD47 ‘Don't Eat Me’ Signal for Macrophages Is Implicated in Immune Evasion

We previously identified CD47 as a ‘don't eat me’ signal for macrophages that, when presented on their surface, enables cancer cells and other pathogenic cells to evade the immune system. CD47 interacts with SIRPα, its receptor on macrophages, to inhibit phagocytosis, and blocking this interaction (e.g. with anti-CD47 antibodies) releases this inhibition and promotes the clearance of harmful cells by macrophage phagocytosis. These findings led to the clinical development in our lab of anti-CD47 as cancer immunotherapy^{11,14}. This approach has at least one clear advantage over chemotherapy, which targets rapidly mutating, changing and diverse cancer cells, leading to a partial response and almost invariably selecting for resistant clones. In contrast, targeting macrophages, whose genome is stable, and unleashing their immune-surveillance potential is expected to yield a more reliable and consistent response as long as the macrophages recognize their targets and remain active.

Macrophage checkpoint inhibition has been pioneered by our group for cancer immunotherapy and is showing promising results in early clinical trials¹⁵⁻¹⁷. In analogy to chemotherapy and cancer, relying only on antibiotics for resolving bacterial infections carries similar inherent limitations due to selection of antibiotic-resistant strains. We hypothesized that, similar to cancer cells, to establish a successful infection, bacteria can develop mimics of the dominant ‘don't eat me’, CD47, and be selected by their avoidance of innate immune responses mediated by macrophage phagocytosis. It is unlikely that bacteria have the similar genes or encoded proteins to mammals' CD47, but is plausible that, by convergent evolution, bacteria would have developed molecules on their surface which mimic the shape of human CD47.

We engineered a variant protein we call CV1 (SEQ ID NO:1) that mimics SIRPα, but binds native CD47 50,000-fold tighter¹². This was engineered to detect the shape of CD47 based on its resolution by x-ray crystallography. We made a fusion between CV1 and the human immunoglobulin G4 heavy chain, yielding a divalent, high-affinity CD47-detecting reagent we call CV1-G4 (SEQ ID NO:2). Using CV1-G4, we identified a cross-reacting bacterial protein on the surface of Bb that, when blocked, promotes macrophage-mediated Bb clearance.

In some embodiments the SIRPα-based blocking agents will block the CD47 mimic on pathogens and will also block the mammalian CD47 and therefore facilitate removal of both the infectious pathogens and of cells already infected with the pathogen that have up-regulated endogenous CD47. The clinical application of CV1-G4 may therefore lead to a combined blockade of CD47 on infected cells, and its mimic on the infecting pathogens.

### Example 2

### Identification of a Bacterial Protein that Functions as a CD47 Mimic, and Its Involvement In Bb Infection and Pathogenesis

We previously identified CD47 as a ‘don't eat me’ signal for macrophages that is presented on the surface of cancer and other pathogenic cells and enables their immune evasion. This mechanism of immune evasion is also used by non-pathogenic cells, which transiently increase their surface CD47 levels to protect themselves in scenarios in which they become vulnerable to phagocytosis (e.g. mobilized hematopoietic stem cells induce CD47 as they exit the bone marrow, where they have to cross fields of macrophages)⁸. We hypothesized that a successful persistent bacterial infection may utilize similar means to inhibit encountering macrophages.

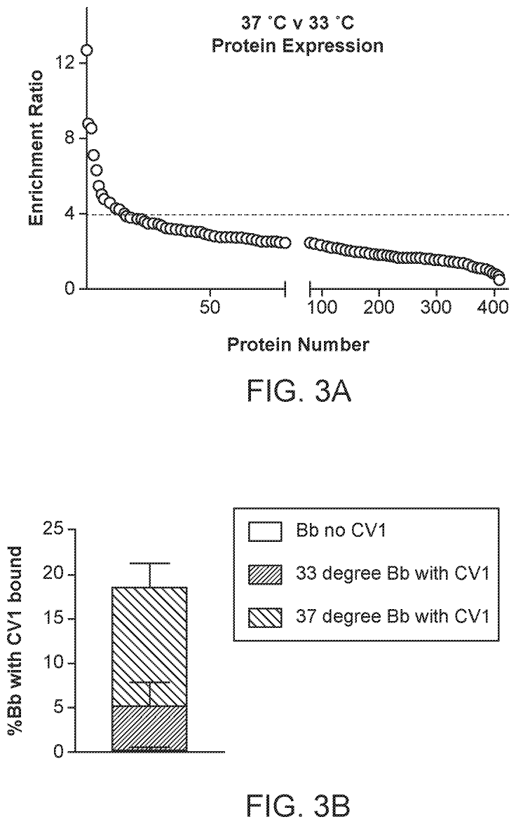

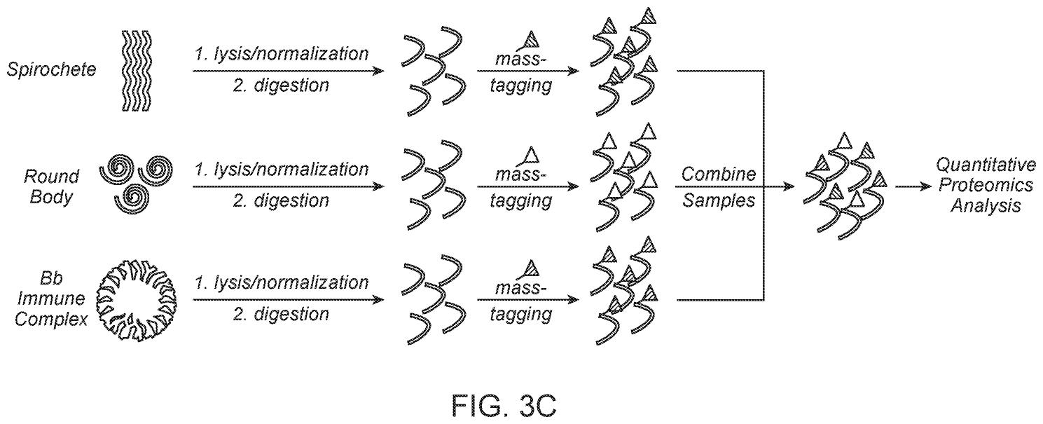

Using the engineered CV1 -G4 protein that binds human CD47 with very high affinity and specificity, we identified a cross-reacting bacterial protein on the surface of Bb that, when blocked, promotes macrophage-mediated Bb clearance. Importantly, we also found that treatment with CV1-G4 blocks the interaction of the bacterial CD47-like protein with SIRPα, the complementary inhibitory receptor on macrophages, thus increasing phagocytosis (FIG. 3A). However, no protein in the Bb proteome aligns with the amino acid sequence of human CD47, suggesting that the similarity is in structure rather than sequence. Importantly, expression of this CD47 mimic is upregulated in response to increased temperature (37° C. compared to 25° C.), suggesting a potential mechanism whereby this protein is induced upon transmission from the tick to the mammal, enabling the pathogen to evade the innate immune system by blocking macrophage-mediated bacterial clearance once in the host's body.

We employed a mass spectrometry-based approach to identify the CD47 mimic. As shown in FIG. 3, we performed immunoprecipitations using CV1-G4 or a control antibody, IgG4, from Bb protein lysates recovered under non-denaturing conditions from bacteria grown at 37° C. or 25° C. Enriched proteins were separated by molecular weight by SDS-PAGE. Gel bands containing proteins that are predominantly enriched with CV1-G4 and not IgG4 at 37° C. or with either antibody at 25° C. were excised, processed and subjected to mass spectrometry analysis.

Under certain conditions, infection by Bb can lead to an allergic response mediated by Bb-specific IgE antibodies, Furthermore, coating the Bb exposed to CHO media with CV1-G4 to block their immunomodulatory CD47 mimic significantly reduced the resultant IgE response and augmented the production of the murine isotype that is intended to clear bacterial infections, IgG2a. However, we found that CV1-G4 as a single-agent therapy is sometimes insufficient to clear an established in vivo infection. To further evaluate potential efficacy, we are testing CV1-G4 in combination with other therapies in late stages when standard doxycycline treatment is no longer effective.

### Example 3

### Therapeutic Targeting of CD47-SIRPα Signaling to Induce Immune Clearance of Persistent Intracellular or Aggregated B. burgdorferi

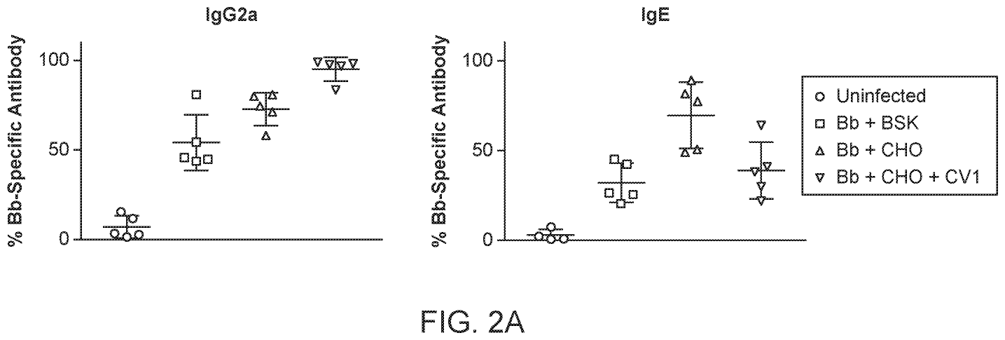

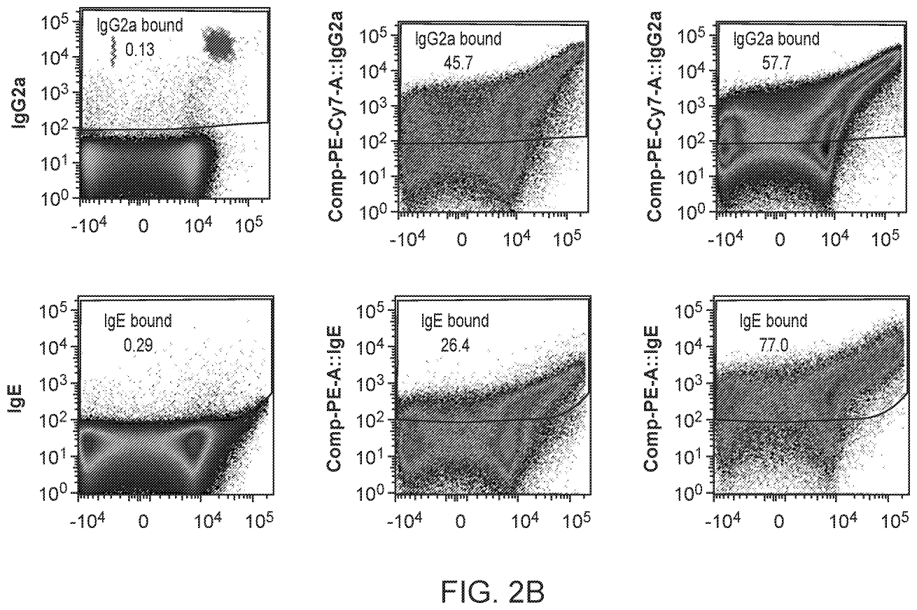

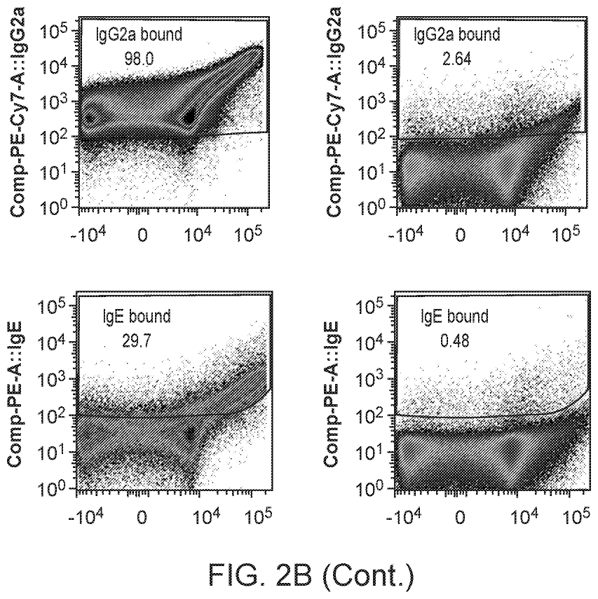

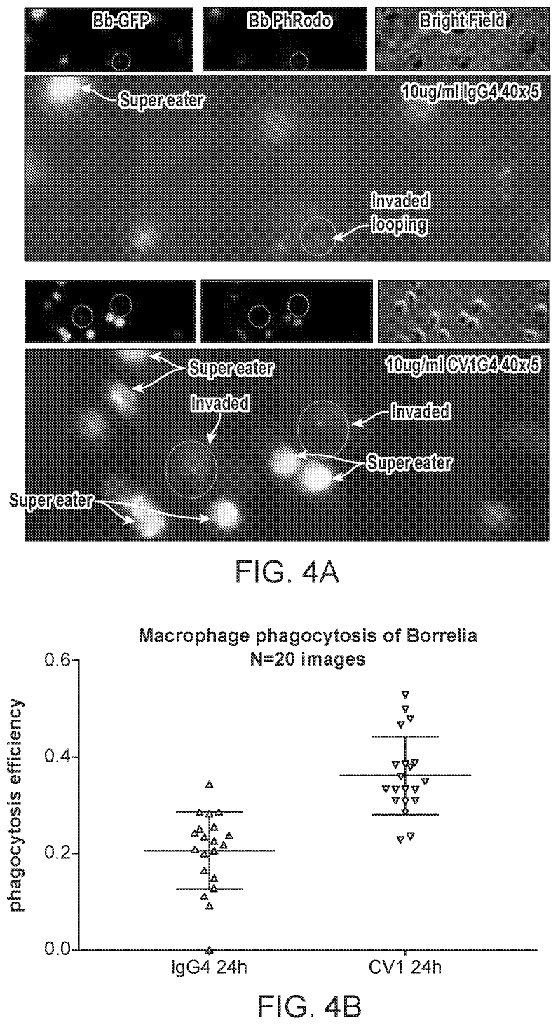

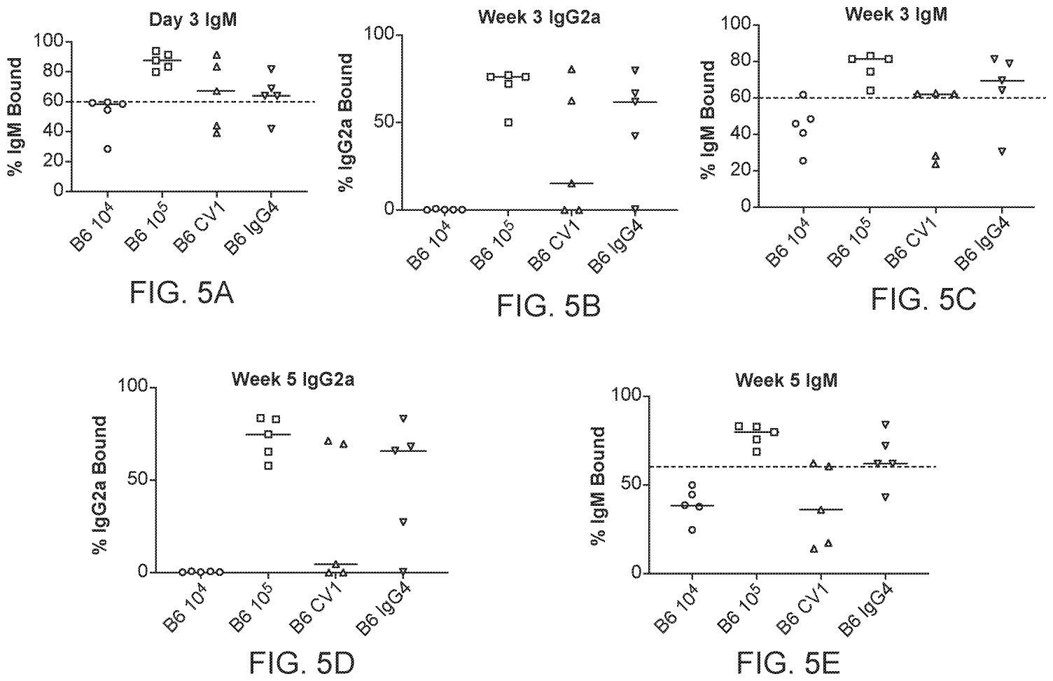

We find that Bb can be found inside cells either by invasion or by phagocytosis. In order to distinguish between these two processes, we double-labeled Bb-GFP with pHrodo—a stain which fluoresces bright red in a low-pH environment such as the lysosome. Thus, phagocytosed Bb will have red fluorescence in addition to GFP. Using this method, we observe that CD47 blockade with CV1-G4 significantly increases phagocytosis of Bb by human macrophages in comparison to treating the culture with the IgG4 isotype control (FIG. 2). Using differential staining of Bb-GFP that is attached to, but not inside of, cells, we find that invading Bb often forms a loop. This behavior has also been observed in the closely-related bacteria that causes Syphilis. Interestingly, Bb in cells that have been treated with CV1-G4 do not appear to form loops, even when they only fluoresce in green (and thus have not been exposed to the acidic lysosome) (FIG. 4). We coated Bb-GFP in CV1-G4 or in an IgG4 isotype control prior to injecting these bacteria intraperitoneally in either C57BL/6 or C3H/HeJ mice at a concentration of 10⁵. We compared these to Bb -GFP injected at 10⁵ or 10⁴ with no antibody coating. We find that BL/6 mice are not infected by 10⁴ bacteria, but are infected with 10⁵ spirochetes (FIG. 5). IgG4 coating of the Bb prevented infection in 1 out of 5 mice, and CV1-G4 coating prevented infection in 3 out of 5 mice, suggesting that the CD47 mimic is important for infection and that blockade with CV1-G4 augments immune clearance both in vitro and in vivo. We find that the number of Bb-GFP required to infect C3H mice is considerably lower, as the 10⁴ injection infected 4 out of 5 mice. CV1-G4 coating of Bb-GFP did not prevent infection at the 10⁵ Bb-GFP dose. We think that this strain-dependent difference in the minimum required dose for infection stems from immune differences in these mouse strains.

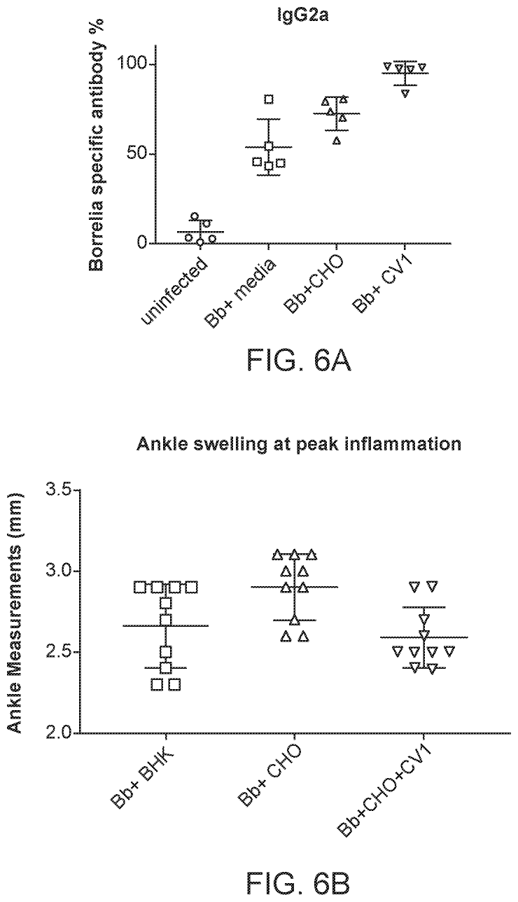

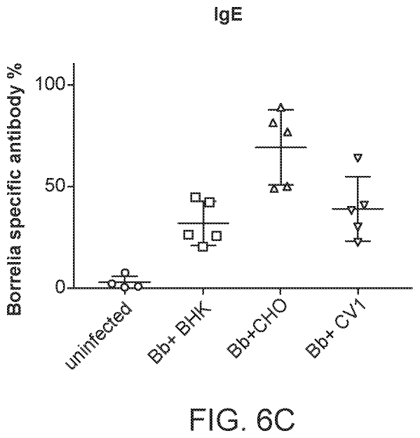

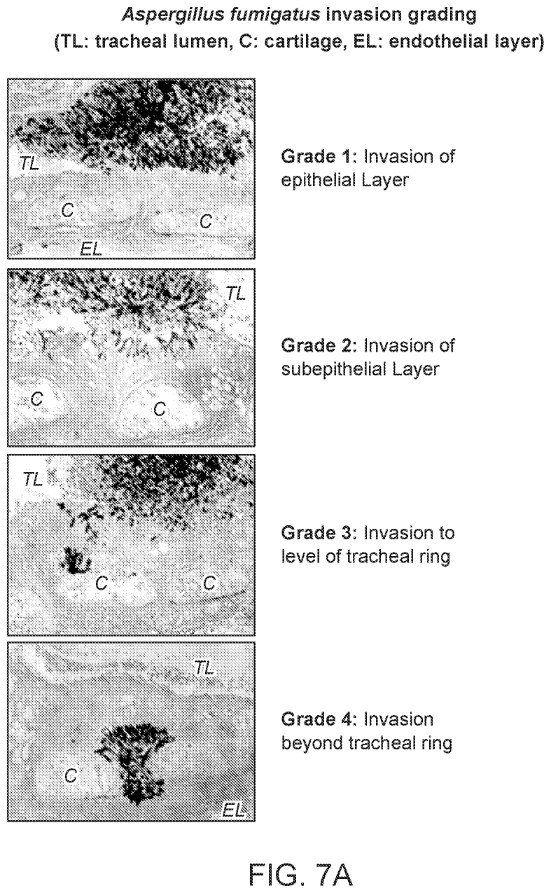

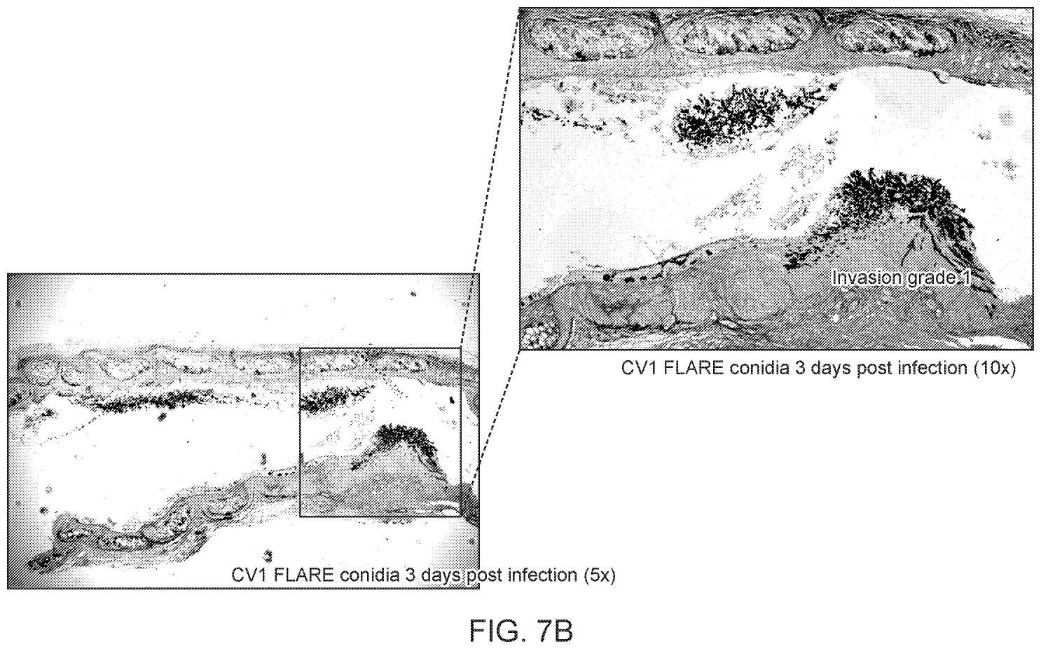

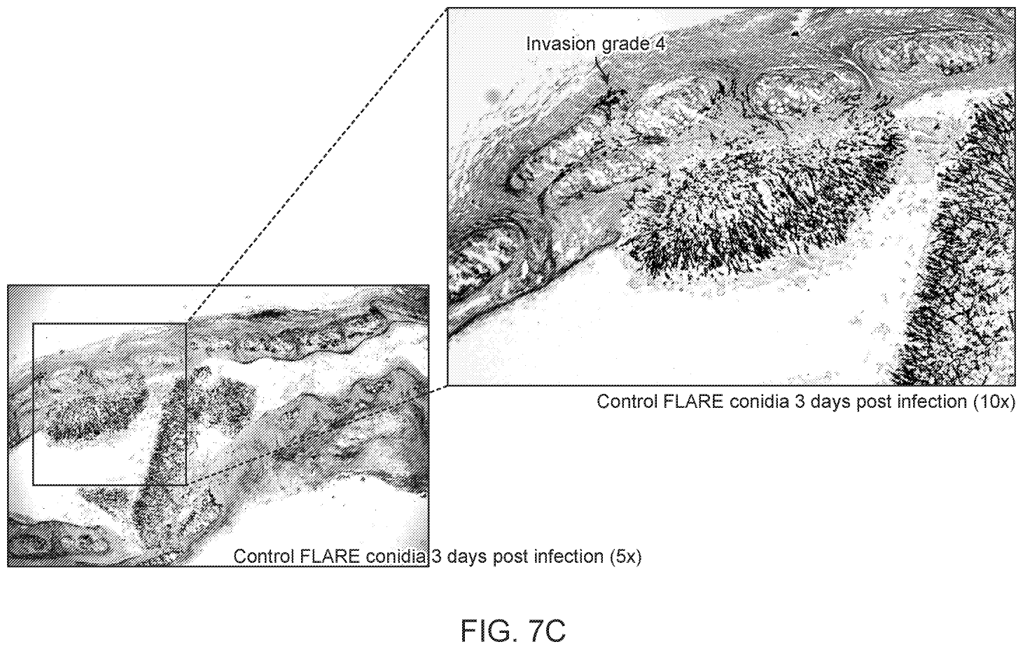

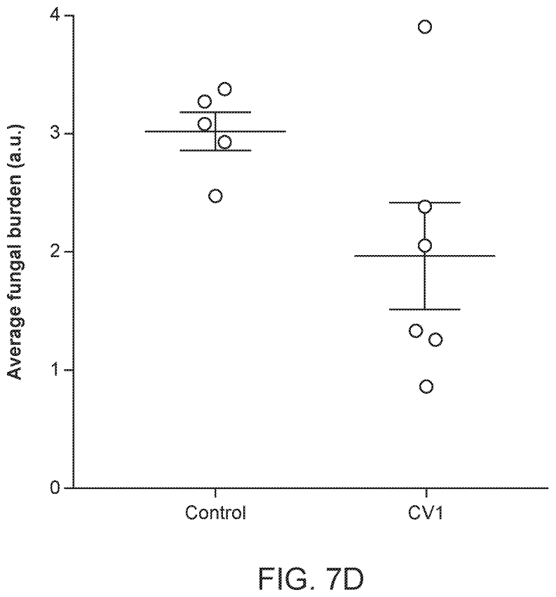

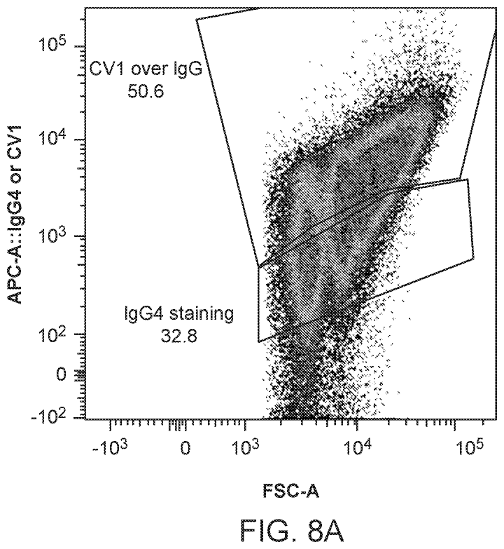

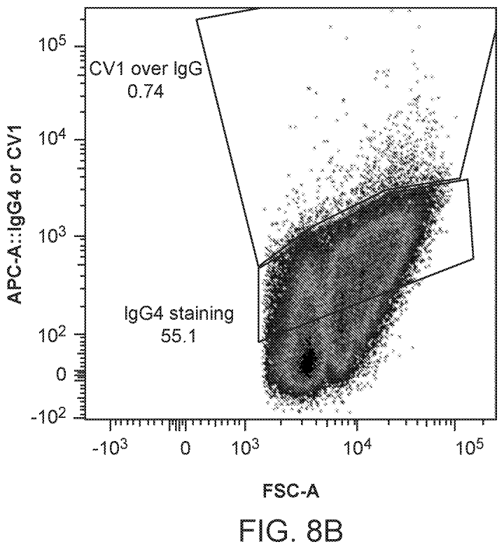

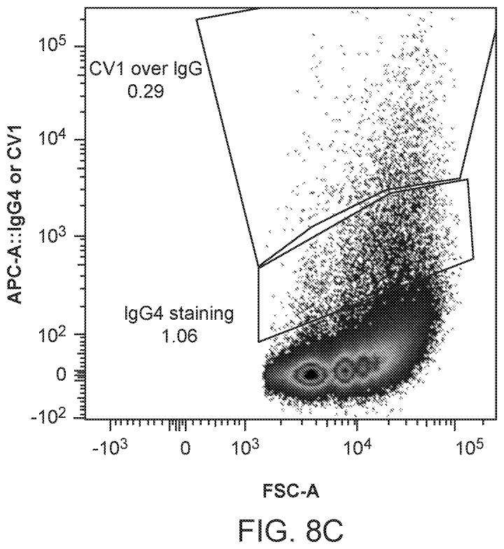

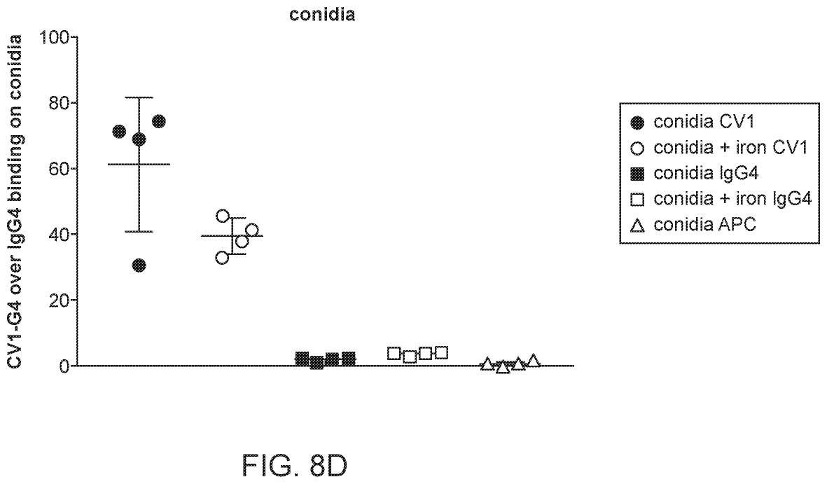

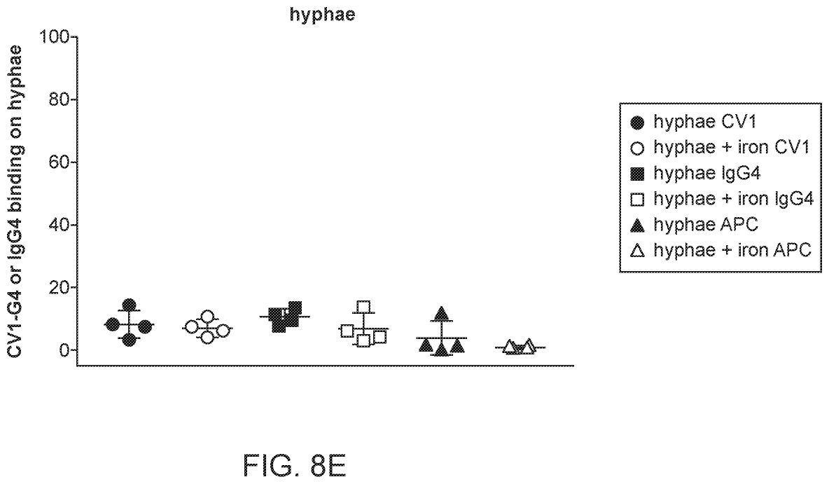

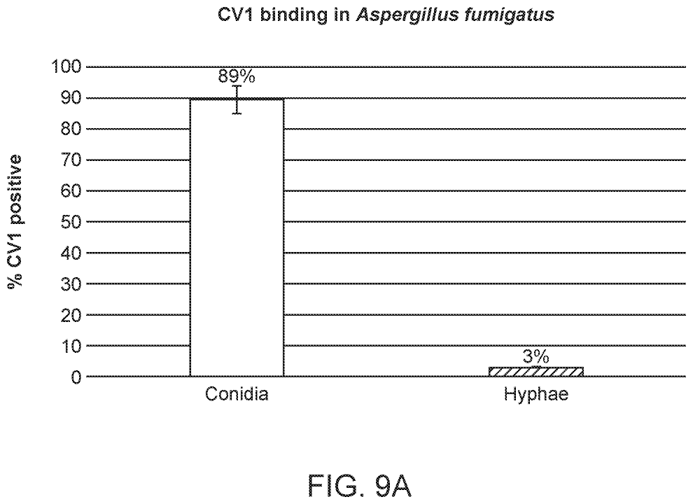

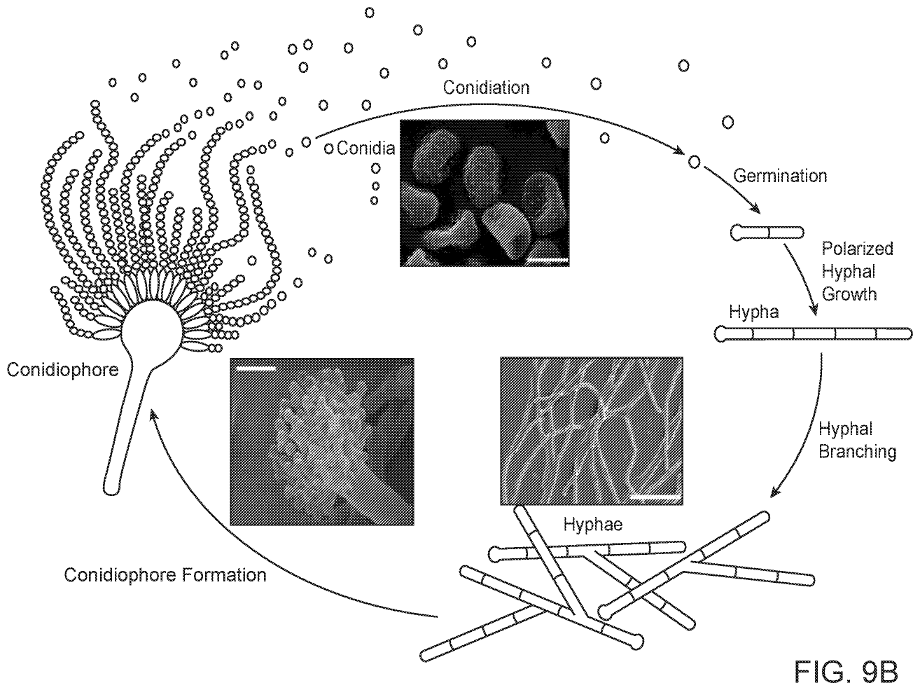

We demonstrated that injection of Bb that have been stressed by incubation in CHO media (a mammalian cell media) induced a much higher Bb-specific IgE response. Furthermore, coating the Bb exposed to CHO media with CV1-G4 to block their immunomodulatory CD47 mimic significantly reduced the resultant IgE response and augmented the production of the ideal murine isotype for clearing bacterial infections, IgG2a (FIG. 6). However, we found that CV1-G4 as a single-agent therapy was insufficient to clear an established in vivo infection. We plan to test CV1-G4 in combination with other therapies in late stages when standard doxycycline treatment is no longer effective. We will infect BL/6 and C3H mice with Bb and treat with doxycycline either together with or followed by CV1-G4 to activate macrophage clearance of Bb and infected cells.

### Example 4

### Identifying Additional CD47 Mimic Proteins Encoded by Bacterial and Fungal Pathogens

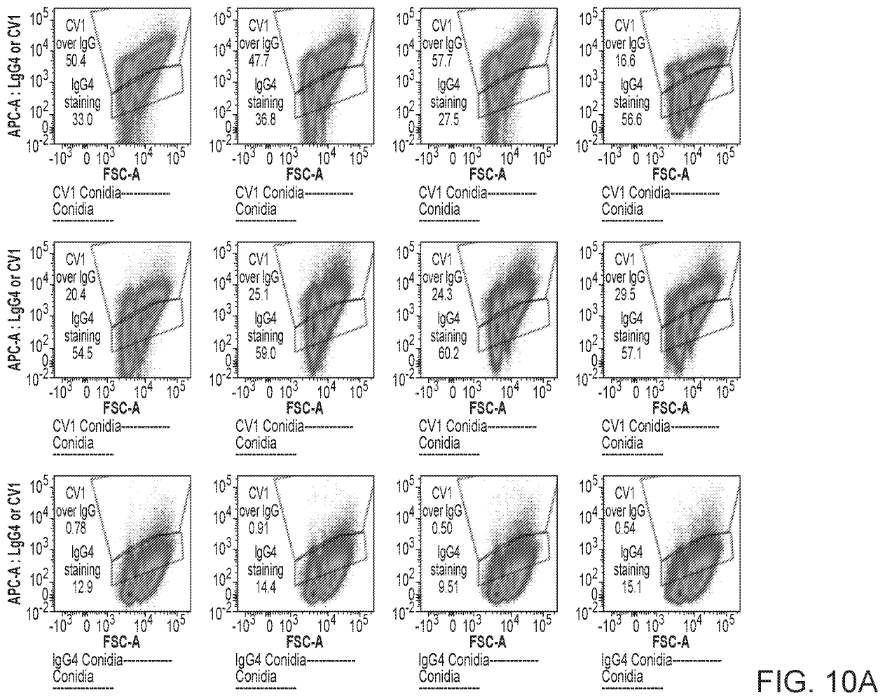

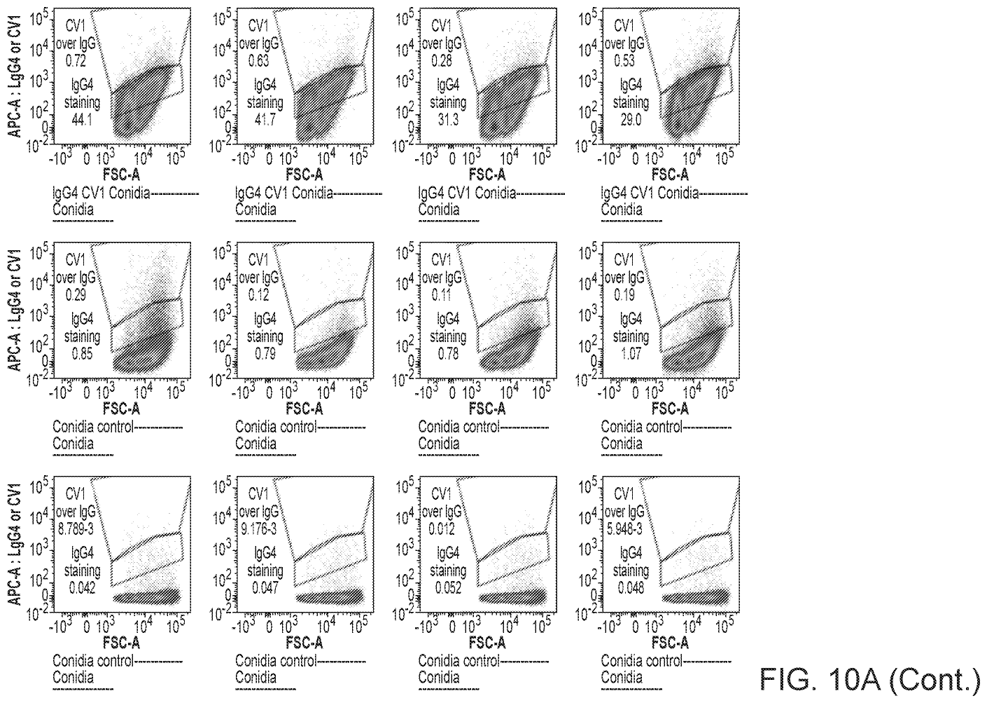

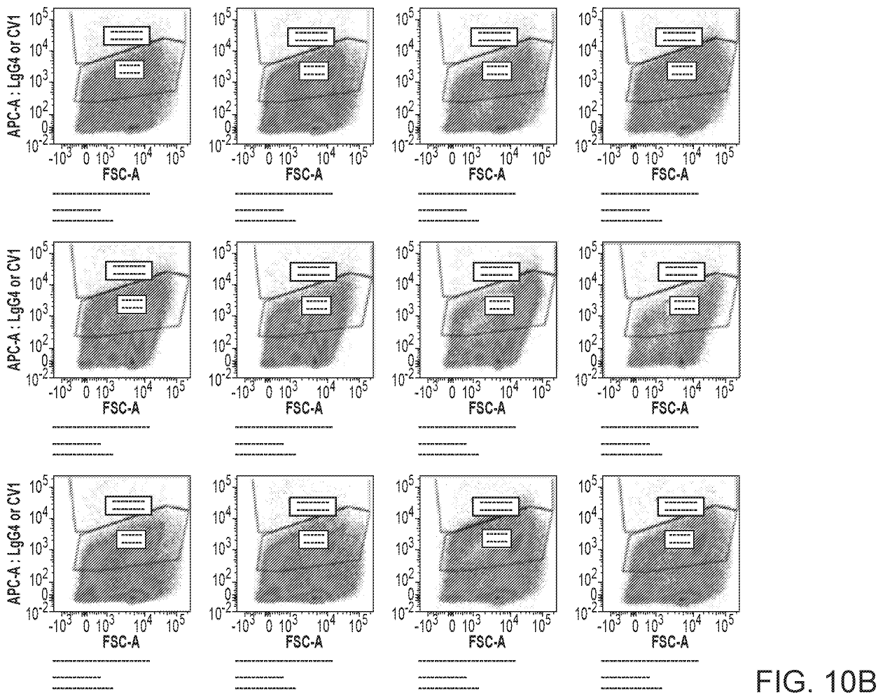

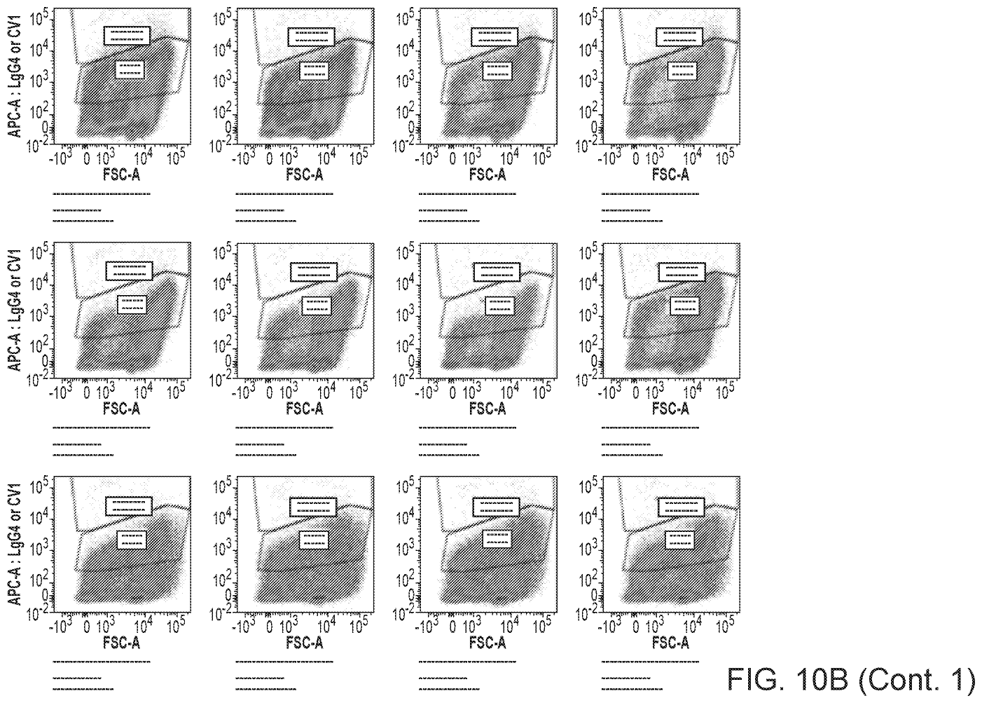

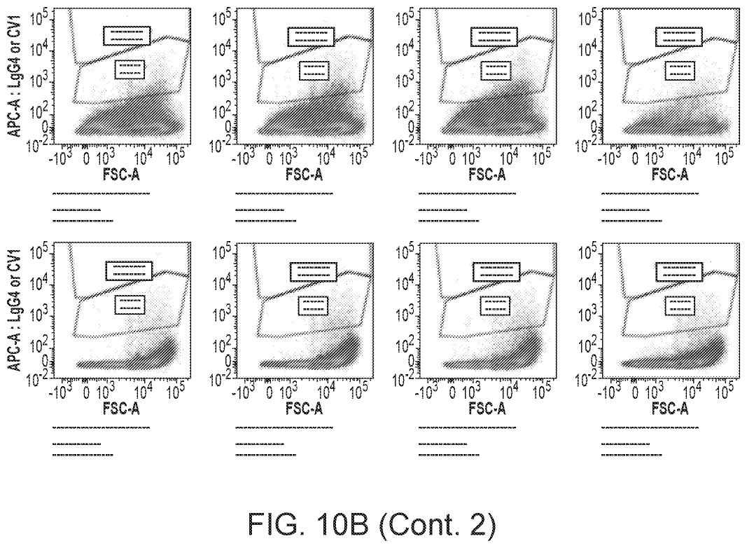

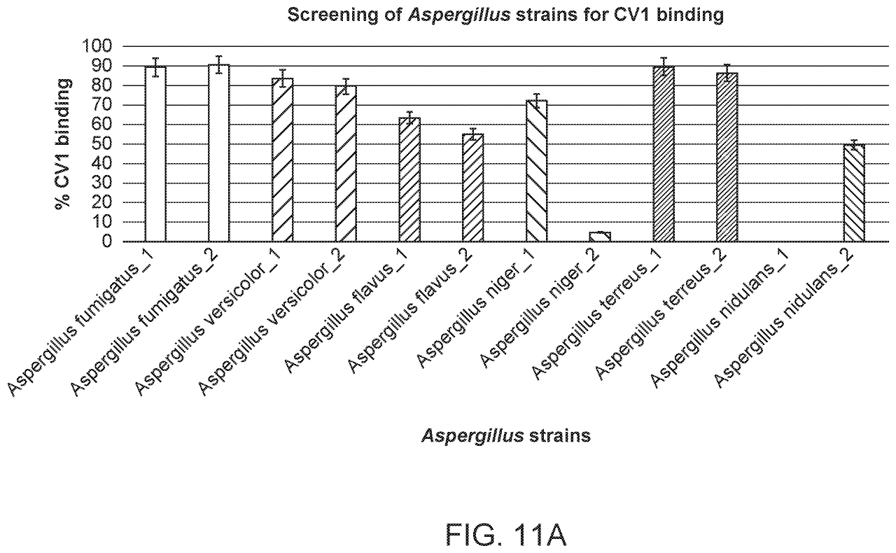

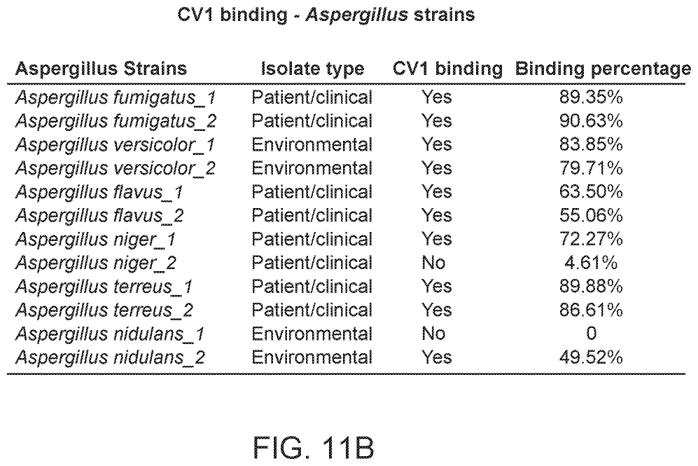

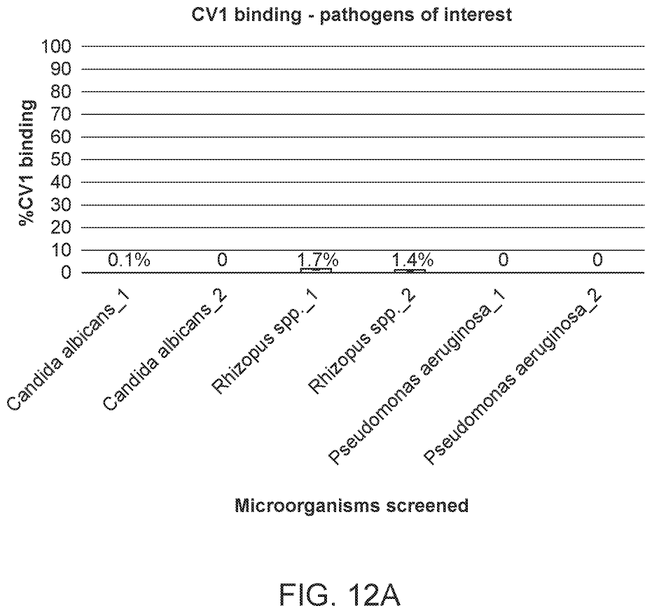

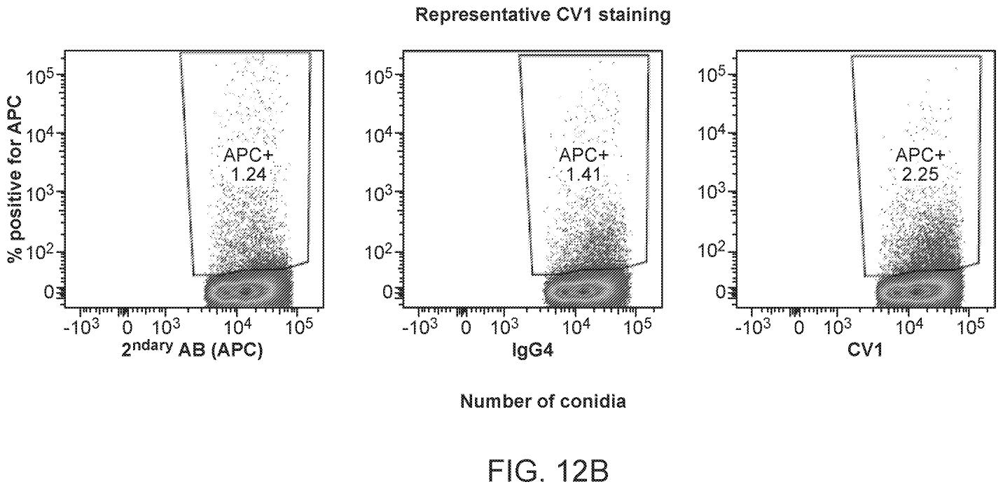

Using the engineered protein CV1-G4, which has very high affinity for CD47, we found and characterized additional CD47 mimic proteins encoded by bacterial and fungal pathogens which can modulate immune responses. We demonstrated CV1 binding to various Aspergillus strains (FIGS. 11A, 11B). We further showed that in Aspergillus fumigatus, CV1-G4 binds to conidia (spores) but not hyphae (FIGS. 10A, 10B). As Aspergillus grows into hyphae, it loses expression of the CD47 mimic that is binding to CV1-G4.

### REFERENCES

1. Feria-Arroyo, T. P. et al. Implications of climate change on the distribution of the tick vector Ixodes scapularis and risk for Lyme disease in the Texas-Mexico transboundary region. Parasit. Vectors 7,199 (2014).

2. Branda, J. A. et al. Advances in Serodiagnostic Testing for Lyme Disease Are at Hand. Clin. Infect. Dis. VIEWPOINTS • CID 2018, 1133

3. Marques, A. R. Laboratory diagnosis of Lyme disease: advances and challenges. Infect. Dis. Clin. North Am. 29, 295-307 (2015).

4. CDC Lyme Disease. Available at: https://www.cdc.gov/lyme/index.html.

5. Trollmo, C., Meyer, A. L., Steere, A. C., Hafler, D. A. & Huber, B. T. Molecular Mimicry in Lyme Arthritis Demonstrated at the Single Cell Level: LFA-1 L Is a Partial Agonist for Outer Surface Protein A-Reactive T Cells. J. Immunol. 166, 5286-5291 (2001).

6. Thangam, E. B. et al. The Role of Histamine and Histamine Receptors in Mast Cell-Mediated Allergy and Inflammation: The Hunt for New Therapeutic Targets. Front. Immunol. 9, 1873 (2018).

7. Hatziagelaki, E., Adamaki, M., Tsilioni, I., Dimitriadis, G. & Theoharides, T. C. Myalgic Encephalomyelitis/Chronic Fatigue Syndrome—Metabolic Disease or Disturbed Homeostasis due to Focal Inflammation in the Hypothalamus? J. Pharmacol. Exp. Ther. 367, 155-167 (2018).

8. Jaiswal, S. et al. CD47 is upregulated on circulating hematopoietic stem cells and leukemia cells to avoid phagocytosis. Cell 138, 271-85 (2009).

9. Majeti, R. et al. CD47 is an adverse prognostic factor and therapeutic antibody target on human acute myeloid leukemia stem cells. Cell 138, 286-99 (2009).

10. Lagasse, E. & Weissman, I. L. bcl-2 inhibits apoptosis of neutrophils but not their engulfment by macrophages. J. Exp. Med. 179, 1047-52 (1994).

11. Willingham, S. B. et al. The CD47-signal regulatory protein alpha (SIRPα) interaction is a therapeutic target for human solid tumors. Proc. Natl. Acad. Sci. U. S. A. 109, 6662-7 (2012).

12. Weiskopf, K. et al. Engineered SIRPα variants as immunotherapeutic adjuvants to anticancer antibodies. Science 341, 88-91 (2013).

13. Chao, M. P., Majeti, R. & Weissman, I. L. Programmed cell removal: a new obstacle in the road to developing cancer. Nat. Rev. Cancer 12, 58-67 (2012).

14. Chao, M. P. et al. Anti-CD47 antibody synergizes with rituximab to promote phagocytosis and eradicate non-Hodgkin lymphoma. Cell 142, 699-713 (2010).

15. Advani, R. et al. CD47 Blockade by Hu5F9-G4 and Rituximab in Non-Hodgkin's Lymphoma. N. Engl. J. Med. 379, 1711-1721 (2018).

16. Takimoto, C. H. et al. The Macrophage ‘Do not Eat Me’ Signal, CD47, Is A Clinically Validated Cancer Immunotherapy Target. Ann. Oncol. (2019). doi:10.1093/annonc/mdz006

17. Sikic, B. I. et al. First-in-Human, First-in-Class Phase I Trial of the Anti-CD47 Antibody Hu5F9-G4 in Patients With Advanced Cancers. J. Clin. Oncol. JC01802018 (2019). doi:10.1200/JCO.18.02018

18. Kenedy, M. R., Lenhart, T. R. & Akins, D. R. The role of Borrelia burgdorferi outer surface proteins. FEMS Immunol. Med. Microbiol. 66, 1-19 (2012).

What is claimed is:

1. A method of inhibiting an infection of a subject by a pathogen comprising a pathogenic CD47 mimic protein, the method comprising administering an effective amount of an agent that reduces binding of the CD47 mimic protein on the pathogen to a signal regulatory protein α (SIRPα) on a phagocytic cell.

2. The method of claim 1, wherein the effective amount of the agent is sufficient to increase phagocytosis of a Borrelia or Aspergillus pathogen by a phagocytic cell.

3. The method of claim 1 or 2, wherein the phagocytic cell is a macrophage.

4. The method of any of claims 1-3, wherein the agent is a SIRPα polypeptide, a soluble CD47, an anti-CD47 mimic antibody, or an anti-SIRPα antibody.

5. The method of claim 4, wherein the SIRPα polypeptide is CV1 or FD6.

6. The method of claim 4, wherein the agent specifically binds to the CD47 mimic protein.

7. The method of any of claims 4-6, wherein the SIRPα polypeptide is fused to an Fc domain of an antibody.

8. The method of claim 7, wherein the antibody is selected from the group consisting of IgG1, IgG2, IgG3, IgG4, IgA, IgE, IgD, and IgM.

9. The method of any of claims 1-8, wherein the pathogen is a Borrelia or Aspergillus pathogen.

10. The method of claim 9, wherein the Borrelia pathogen is Borrelia burgdorferi.

11. The method of claim 9, wherein the Aspergillus pathogen is selected from the group consisting of Aspergillus fumigatus, Aspergillus versicolor, Aspergillus flavus, Aspergillus niger, Aspergillus terreus, and Aspergillus nidulans.

12. The method of claim 11, wherein the agent binds to the CD47 mimic protein on conidia.

13. A method of treating a subject for a Borrelia or Aspergillus infection associated with production of a pathogenic CD47 mimic protein, the method comprising administering a therapeutically effective amount of an agent that binds to the CD47 mimic protein to the subject.

14. The method of claim 13, wherein the Borrelia infection is caused by Borrelia burgdorferi.

15. The method of claim 13, wherein the Aspergillus infection is caused by an Aspergillus pathogen selected from the group consisting of Aspergillus fumigatus, Aspergillus versicolor, Aspergillus flavus, Aspergillus niger, Aspergillus terreus, and Aspergillus nidulans.

16. The method of claim 15, wherein the agent binds to the CD47 mimic protein on conidia.

17. The method of any of claims 13-16, wherein the agent is conjugated to a cytotoxic agent, an antibacterial agent, or an antifungal agent.

18. The method of any of claims 13-17, wherein the agent is a SIRPα polypeptide or an antibody that binds to the CD47 mimic protein.

19. The method of claim 18, wherein the SIRPα polypeptide comprises CV1 or FD6.

20. The method of claim 18 or 19, wherein the SIRPα polypeptide is fused to an Fc domain of an antibody.

21. The method of claim 20, wherein the antibody is selected from the group consisting of IgG1, IgG2, IgG3, IgG4, IgA, IgE, IgD, and IgM.

22. A method of detecting a Borrelia or Aspergillus pathogen expressing a pathogenic CD47 mimic protein, the method comprising:
a) contacting the CD47 mimic protein on the Borrelia or Aspergillus pathogen with a probe comprising detectably labeled CV1-G4, wherein the detectably labeled CV1-G4 binds to the CD47 mimic protein to form a complex; and
b) detecting a signal from the detectably labeled CV1-G4 in the complex.

23. The method of claim 22, wherein the detectably labeled CV1-G4 comprises a detectable label selected from the group consisting of a fluorophore, a chemiluminescent label, a bioluminescent label, an isotopic label, and a contrast agent.

24. The method of claim 22 or 23, wherein the method is performed in vivo or in vitro.

25. The method of any of claims 22-24, wherein the probe is immobilized on a solid support.

26. The method of claim 25, wherein the solid support is a magnetic bead, a non-magnetic bead, a membrane, or a gel.

27. The method of any of claims 22-26, further comprising isolating the pathogen from the complex.

28. The method of any of claims 22-27, wherein the Borrelia pathogen is Borrelia burgdorferi.

29. The method of any of claims 22-27, wherein the Aspergillus pathogen is selected from the group consisting of Aspergillus fumigatus, Aspergillus versicolor, Aspergillus flavus, Aspergillus niger, Aspergillus terreus, and Aspergillus nidulans.
